(19) United States
(12) Patent Application Publication
(10) Pub. No.: US 2024/0386553 A1
(43) Pub. Date: Nov. 21, 2024

(54) DOMAIN ADAPTATION TO ENHANCE IVUS IMAGE FEATURES FROM OTHER IMAGING MODALITIES

(71) Applicant: Boston Scientific Scimed Inc., Maple Grove, MN (US)

(72) Inventors: Hatice Cinar Akakin, Eden Prairie, MN (US); Kevin Bloms, Minneapolis, MN (US); Wenguang Li, Los Gatos, CA (US)

(73) Assignee: Boston Scientific Scimed Inc., Maple Grove, MN (US)

(21) Appl. No.: 18/666,470

(22) Filed: May 16, 2024

Related U.S. Application Data
(60) Provisional application No. 63/502,809, filed on May 17, 2023.

(51) Int. Cl. G06T 7/00; G06V 10/44; G16H 30/40

(52) U.S. Cl. CPC G06T 7/0012; G06V 10/44; G16H 30/40; G06T 2207/10132; G06T 2207/20084; G06T 2207/30101

(57) ABSTRACT

The present disclosure provides devices and methods to process intravascular images of a vessel of one imaging modalities and to generate, extract and adapt features from another imaging modality to generate a hybrid image comprising features from both modalities. The disclosure provides devices and methods to train deep generative models to adapt domain specific features from one intravascular imaging modality (e.g., OCT, or the like) to another intravascular imaging modality (e.g., IVUS) and integrate the adapted features into the images from the other intravascular imaging modality.

### CROSS-REFERENCE TO RELATED APPLICATIONS

This application claims the benefit of U.S. Provisional Patent Application Ser. No. 63/502,809 filed on May 17, 2023, the disclosure of which is incorporated herein by reference.

### TECHNICAL FIELD

The present disclosure generally relates to intravascular ultrasound (IVUS) imaging system. Particularly, but not exclusively, the present disclosure relates to enhancing features of IVUS images based on domain adaptation from other imaging modalities (e.g., optical coherence tomography, or the like).

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

IVUS imaging systems include a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

There are other endoluminal imaging modalities, such as, for example, optical coherence tomography (OCT), which uses infrared light instead of ultrasound to capture intravascular images. In some procedures, a “hybrid” catheter can be used where the hybrid catheter can capture both IVUS images and OCT images in the same procedure.

There is a need to utilize both imaging modalities in a complimentary way to produce better clinical outcomes.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides to adapt features from one imaging modality (e.g., OCT) to enhance the features of another imaging modality (e.g., IVUS) to reduce the need to screen against multiple imaging modalities. The disclosure provides to utilize machine learning (ML) such as deep generative models (Generative Adversarial Networks, Generative Diffusion Models, etc.) to adapt domain specific features from one intravascular imaging modalities (e.g., OCT, or the like) to another intravascular imaging modality (e.g., IVUS) and integrate the adapted features into the IVUS images.

As such, the present disclosure provides an advantage over conventional techniques, systems, and treatments in that increased clinical outcomes can be achieved based on improved diagnostic capabilities (e.g., diagnosing lesions, or the like) or for improved procedure guidance (e.g., percutaneous coronary intervention (PCI) guidance, or the like). Further, as stated, the present disclosure provides a reduction in the number of imaging modalities that need be screened to treat a patient and/or analyze treatment results.

In some embodiments, the disclosure can be implemented as a method for a computing device. The method can comprise receiving, at a processor, a first series of intravascular images of a vessel of a patient, the first series of intravascular images of a first imaging modality; generating, by the processor from the first series of intravascular images, image features of a second imaging modality; enhancing, by the processor, the first series of intravascular images with the image features of the second series imaging modality; and generating, by the processor, a graphical user interface comprising an indication of the enhanced first series of intravascular images.

With further embodiments, the method can comprise causing the graphical user interface to be displayed on a display coupled to the computing device.

With further embodiments of the method, generating the image features of a second imaging modality can comprise generating, via a machine learning (ML) model, a second series of intravascular images of the vessel of the patient, the second series of intravascular images of the second imaging modality; and generating, via the ML model, the image features of the second imaging modality from the first series of intravascular images.

With further embodiments of the method, generating the image features of the second imaging modality from the first series of intravascular images can comprise translating, via the ML model, the first series of intravascular images to the second imaging modality to form a series of translated intravascular images, wherein the series of translated intravascular image features look like image features of the second imaging modality; and extracting, via the ML model, features from the series of translated images.

With further embodiments of the method, enhancing the first series of intravascular images with the image features of the second imaging modality can comprise generating, via the ML model, a series of hybrid intravascular images comprising the first series of intravascular images and the image features of the second imaging modality.

With further embodiments of the method, the ML model comprises a medical image generative network and auxiliary task networks, wherein the auxiliary task networks are arranged to preserve the geometry of extracted features.

With further embodiments of the method, the ML model is trained using a plurality of series of intravascular images of the first modality paired or unpaired with a respective series of a plurality of series of intravascular images of the second modality.

With further embodiments of the method, the medical image generative model is trained with non-adversarial loss from the auxiliary task network.

With further embodiments of the method, the ML model comprises a convolutional neural network (CNN) based encoder network and a first decoder network and a second decoder network.

With further embodiments of the method, the CNN based encoder network is arranged to translate a series of intravascular images of the first imaging modality into a series of intravascular images of the second imaging modality and translate a series of intravascular images of the second imaging modality into a series of intravascular images of the first imaging modality.

With further embodiments of the method, the first decoder network is arranged to extract features from the series of intravascular images translated from the first imaging modality.

With further embodiments of the method, the second decoder network is arranged to extract features from the series of intravascular images translated from the second imaging modality.

With further embodiments of the method, the ML model is trained with a plurality of series of intravascular images of the first modality paired or unpaired with a respective series of a plurality of series of intravascular images of the second modality annotated with ground truth masks.

In some embodiments, the disclosure can be implemented as an apparatus, comprising a memory and a processor coupled to the memory and configured to be coupled to an intravascular ultrasound (IVUS) imaging system. The memory can comprise instructions executable by the processor, which instructions when executed cause the processor to implement any of the methods described herein.

In some embodiments, the disclosure can be implemented as a machine readable storage device, comprising a plurality of instructions that in response to being executed by a processor of an intravascular ultrasound (IVUS) imaging system cause the processor to implement any of the methods described herein.

In some embodiments, the disclosure can be implemented as an apparatus, comprising a memory and a processor coupled to the memory and configured to be coupled to an intravascular ultrasound (IVUS) imaging system. The memory can comprise instructions executable by the processor, which instructions when executed cause the processor to receive a first series of intravascular images of a vessel of a patient, the first series of intravascular images of a first imaging modality; generate, from the first series of intravascular images, image features of a second imaging modality; enhance the first series of intravascular images with the image features of the second series imaging modality; and generate a graphical user interface comprising an indication of the enhanced first series of intravascular images.

With some embodiments of the apparatus, the instructions when executed by the processor further cause the processor to cause the graphical user interface to be displayed on a display coupled to the computing device.

With some embodiments of the apparatus, the instructions when executed by the processor further cause the processor to generate, via a machine learning (ML) model, a second series of intravascular images of the vessel of the patient, the second series of intravascular images of the second imaging modality; and generate, via the ML model, the image features of the second imaging modality from the first series of intravascular images.

With some embodiments of the apparatus, the instructions when executed by the processor further cause the processor to translate, via the ML model, the first series of intravascular images to the second imaging modality to form a series of translated intravascular images, wherein the series of translated intravascular image features look like image features of the second imaging modality; and extract, via the ML model, features from the series of translated images.

With some embodiments of the apparatus, the instructions when executed by the processor further cause the processor to generate, via the ML model, a series of hybrid intravascular images comprising the first series of intravascular images and the image features of the second imaging modality.

With some embodiments of the apparatus, the ML model comprises a medical image generative network and auxiliary task networks, wherein the auxiliary task networks are arranged to preserve the geometry of extracted features.

With some embodiments of the apparatus, the ML model is trained using a plurality of series of intravascular images of the first modality paired or unpaired with a respective series of a plurality of series of intravascular images of the second modality.

With some embodiments of the apparatus, the medical image generative model is trained with non-adversarial loss from the auxiliary task network.

With some embodiments of the apparatus, the ML model comprises a convolutional neural network (CNN) based encoder network and a first decoder network and a second decoder network.

With some embodiments of the apparatus, the CNN based encoder network is arranged to translate a series of intravascular images of the first imaging modality into a series of intravascular images of the second imaging modality and translate a series of intravascular images of the second imaging modality into a series of intravascular images of the first imaging modality.

With some embodiments of the apparatus, the first decoder network is arranged to extract features from the series of intravascular images translated from the first imaging modality.

With some embodiments of the apparatus, the second decoder network is arranged to extract features from the series of intravascular images translated from the second imaging modality.

With some embodiments of the apparatus, the ML model is trained with a plurality of series of intravascular images of the first modality paired or unpaired with a respective series of a plurality of series of intravascular images of the second modality annotated with ground truth masks.

In some embodiments, the disclosure can be implemented as a machine readable storage device, comprising a plurality of instructions that in response to being executed by a processor of an intravascular ultrasound (IVUS) imaging system cause the processor to receive, at the processor, a first series of intravascular images of a vessel of a patient, the first series of intravascular images of a first imaging modality; generate, by the processor from the first series of intravascular images, image features of a second imaging modality; enhance, by the processor, the first series of intravascular images with the image features of the second series imaging modality; and generate, by the processor, a graphical user interface comprising an indication of the enhanced first series of intravascular images.

With some embodiments of the machine readable storage device, the instructions when executed by the processor further cause the processor to cause the graphical user interface to be displayed on a display coupled to the computing device.

With some embodiments of the machine readable storage device, the instructions when executed by the processor further cause the processor to generate, via a machine learning (ML) model, a second series of intravascular images of the vessel of the patient, the second series of intravascular images of the second imaging modality; and generate, via the ML model, the image features of the second imaging modality from the first series of intravascular images.

With some embodiments of the machine readable storage device, the instructions when executed by the processor further cause the processor to translate, via the ML model, the first series of intravascular images to the second imaging modality to form a series of translated intravascular images, wherein the series of translated intravascular image features look like image features of the second imaging modality; and extract, via the ML model, features from the series of translated images.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an example intravascular ultrasound (IVUS) system in accordance with at least one embodiment of the present disclosure.

FIG. 2 illustrates an extravascular image.

FIGS. 3A and 3B illustrate intravascular images.

FIG. 4 illustrates a multi-image modality adaptation system in accordance with at least one embodiment of the present disclosure.

FIG. 5 illustrates a logic flow in accordance with at least one embodiment of the present disclosure.

FIG. 6 illustrates a machine learning (ML) system suitable for use with exemplary embodiments of the present disclosure.

FIG. 7 illustrates an example machine learning model architecture in accordance with at least one embodiment of the present disclosure.

FIG. 8 illustrates another example machine learning model architecture in accordance with at least one embodiment of the present disclosure.

FIG. 9 illustrates yet another example of machine learning model architecture in accordance with at least one embodiment of the present disclosure.

FIG. 10 illustrates a computer-readable storage medium.

FIG. 11 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to intravascular imaging modalities (e.g., IVUS, OCT, etc.) that capture images and lumens (e.g., vessels) of patients and to processing these images and to adapting features from one modality and integrating them into another modality. As such, an example intravascular imaging system, patient vessel, and series of intravascular images are described below. It is noted that the example system is described as an IVUS system. However, it will be appreciated that the described system could be an OCT system (or other intravascular imaging modality) and/or a combined non-IVUS/IVUS system without departing from the scope of the disclosure.

Suitable intravascular imaging system, such as an IVUS imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Numbers 2006/0100522; 2006/0106320; 2006/0173350; 2006/0253028; 2007/0016054; and 2007/0038111; all of which are incorporated herein by reference.

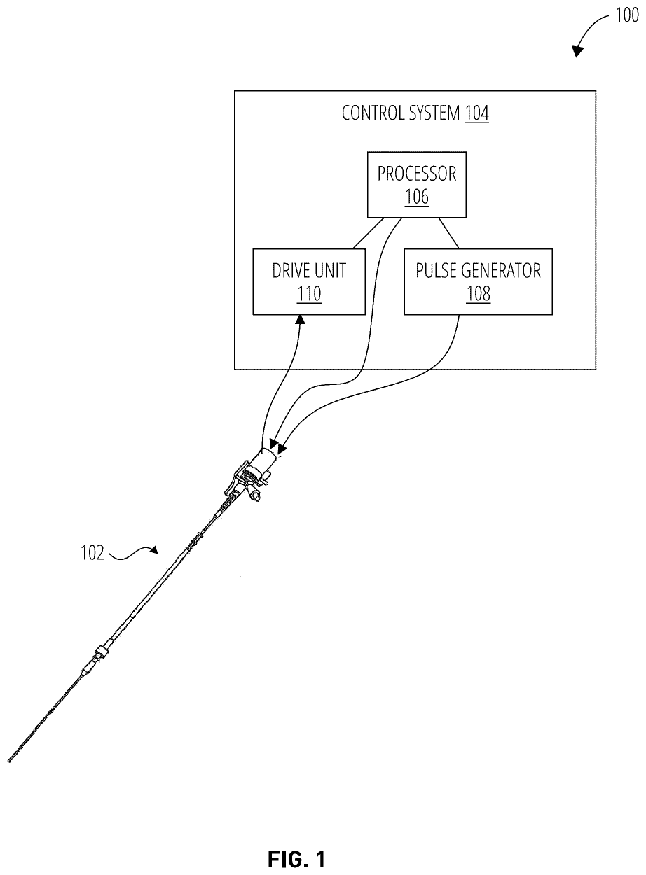

FIG. 1 illustrates schematically one embodiment of an Intravascular imaging systems 100. The Intravascular imaging systems 100 includes a catheter 102 that is couplable to a control system 104. The control system 104 may include, for example, a processor 106, a pulse generator 108, and a drive unit 110. In at least some embodiments, the pulse generator 108 forms electric pulses that may be input to one or more transducers (not shown) disposed in the catheter 102.

With some embodiments, mechanical energy from the drive unit 110 can be used to drive an imaging core (also not shown) disposed in the catheter 102. In at least some embodiments, electric signals transmitted from the one or more transducers may be input to the processor 106 for processing. In at least some embodiments, the processed electric signals from the one or more transducers can be used to form a series of images, described in more detail below. For example, a scan converter can be used to map scan line samples (e.g., radial scan line samples, or the like) to a two-dimensional Cartesian grid, which can be used as the basis for a series of IVUS images that can be displayed for a user.

In at least some embodiments, the processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where Intravascular imaging systems 100 is configured for automatic pullback, the drive unit 110 can control the velocity and/or length of the pullback.

As noted, introduced above, the disclosure relates to adapting features from one imaging modality to another. As such, catheter 102 could be a hybrid catheter arranged to emit and receive both ultrasound and for example, infrared light, to capture both IVUS and OCT type images. Or, in another example, Intravascular imaging systems 100 could be provided with catheter 102 and a different catheter arranged to capture non-IVUS intravascular images (e.g., OCT, or the like).

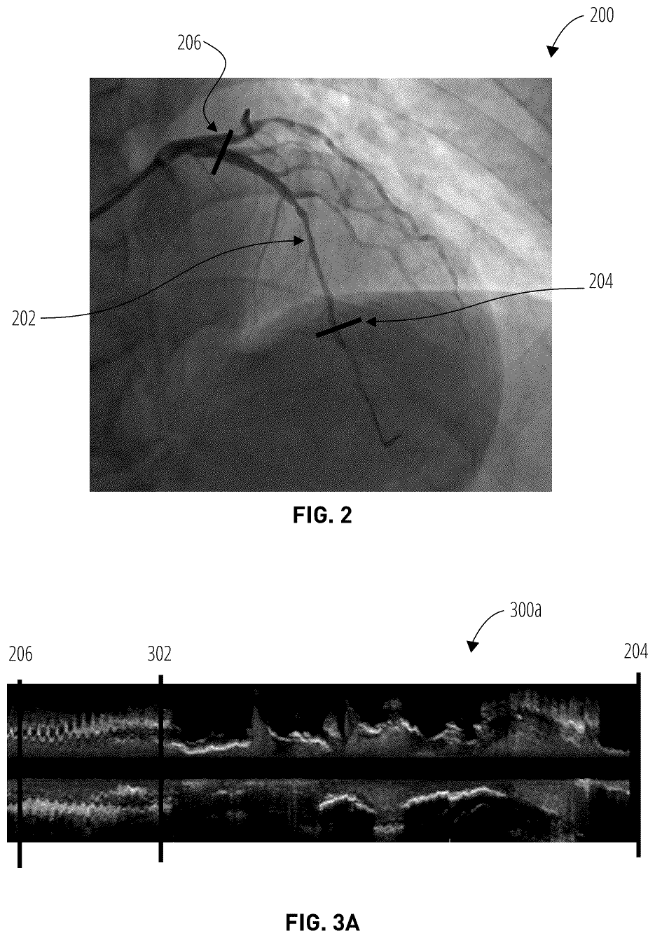

FIG. 2 illustrates an extravascular image 200 of a vessel 202 of a patient. As described, IVUS imaging systems (e.g., Intravascular imaging systems 100, or the like) are used to capture a series of intraluminal images or a “recording” or a vessel, such as, vessel 202. For example, an IVUS catheter (e.g., catheter 102) is inserted into vessel 202 and a recording, or a series of IVUS images, is captured as the catheter 102 is pulled back from a distal end 204 to a proximal end 206. The catheter 102 can be pulled back manually or automatically (e.g., under control of drive unit 110, or the like). The series of IVUS images captured between distal end 204 and proximal end 206 are often referred to an images from an IVUS run.

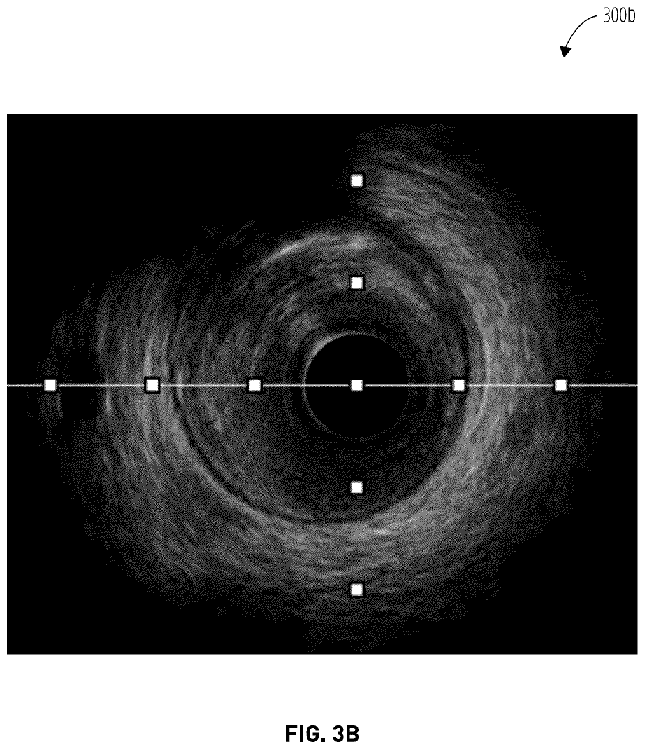

FIG. 3A and FIG. 3B illustrates two-dimensional (2D) representations of IVUS images of vessel 202. For example, FIG. 3A illustrates IVUS images 300a depicting a longitudinal view of the IVUS recording of vessel 202 between proximal end 206 and distal end 204.

FIG. 3B illustrates an image frame 300b depicting an on-axis (or short axis) view of vessel 202 at point 302. Said differently, image frame 300b is a single frame or single image from a series of IVUS images that can be captured between distal end 204 and proximal end 206 as described herein. As introduced above, a physician will often capture an IVUS run (e.g., series of IVUS images) at different stages of treatment. For example, IVUS images may be captured prior to a percutaneous coronary intervention (PCI) treatment and after the PCI treatment (e.g., placement of a stent, balloon dilation, rotablation, or the like) has been performed.

Further, another series of images of vessel 202 can be captured (e.g., either simultaneously with a hybrid catheter, or at another time with a different catheter) using a non-IVUS imaging modality. The present disclosure provides to adapt features from the non-IVUS imaging modality to the IVUS imaging modality and to incorporate the adapted features into the IVUS images. It is to be appreciated that although the description discussed captured adapting non-IVUS imaging features to the IVUS imaging domain the reverse could be implemented based on the present disclosure. For example, IVUs imaging features could be adapted to the OCT domain and OCT images enhanced with the adapted features.

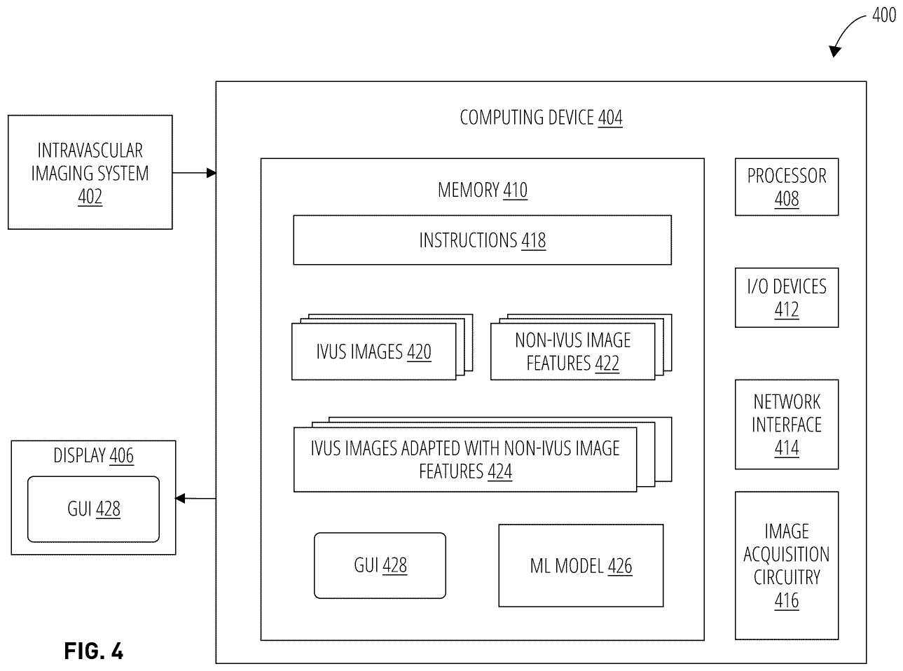

FIG. 4 illustrates a multi-image modality adaptation system 400, according to some embodiments of the present disclosure. In general, multi-image modality adaptation system 400 is a system for adapting images from one intravascular imaging modality with features from images from another imaging modality. Further, multi-image modality adaptation system 400 can be configured to display the adapted images, for example, in a GUI. Multi-image modality adaptation system 400 can be implemented in a commercial IVUS guidance or navigation system, such as, for example, the AVVIGO® Guidance System available from Boston Scientific®. The present disclosure provides advantages over prior or conventional intravascular imaging and guidance systems in that the disclosure provides a composite image comprising indications of features from multiple imaging modalities. For example, the present disclosure can adapt IVUS images, which have a better penetration of a vessel wall and greater vessel morphology than other modalities with features from another modality (e.g., OCT) that are better captured by the other modality (e.g., indications of thickness of calcium and plaque cap, indications of spatial resolution of the lumen of the vessel, or the like). It is noted that the example embodiment in FIG. 4 depicts a system arranged to adapt IVUS images with features from the OCT imaging modality. However, the claims are not limited in this context and a system like the multi-image modality adaptation system 400 could be implemented to adapt OCT images with features from the IVUS imaging modality.

With some embodiments, multi-image modality adaptation system 400 could be implemented as part of control system 104. Alternatively, control system 104 could be implemented as part of multi-image modality adaptation system 400. As depicted, multi-image modality adaptation system 400 includes a computing device 404. Optionally, multi-image modality adaptation system 400 includes intravascular imaging systems 100 and display 406.

Computing device 404 can be any of a variety of computing devices. In some embodiments, computing device 404 can be incorporated into and/or implemented by a console of display 406. With some embodiments, computing device 404 can be a workstation or server communicatively coupled to computing device 404 and/or display 406. With still other embodiments, computing device 404 can be provided by a cloud based computing device, such as, by a computing as a service system accessibly over a network (e.g., the Internet, an intranet, a wide area network, or the like). Computing device 404 can include processor 408, memory 410, input and/or output (I/O) devices 412, network interface 414, and intravascular imaging system acquisition circuitry 416.

Intravascular imaging system 402 can be an intravascular imaging system (e.g., intravascular imaging systems 100) configured to generate intravascular images of a particular imaging modality or could be an intravascular imaging system with a hybrid catheter arranged to generate intravascular images of multiple modalities. In an example embodiment, intravascular imaging system 402 can include an IVUS imaging system (e.g., intravascular imaging systems 100), an OCT imaging system or a hybrid IVUS/OCT imaging system.

The processor 408 may include circuity or processor logic, such as, for example, any of a variety of commercial processors. In some examples, processor 408 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 408 may include graphics processing portions and may include dedicated memory, multiple-threaded processing and/or some other parallel processing capability. In some examples, the processor 408 may be an application specific integrated circuit (ASIC) or a field programmable integrated circuit (FPGA).

The memory 410 may include logic, a portion of which includes arrays of integrated circuits, forming non-volatile memory to persistently store data or a combination of non-volatile memory and volatile memory. It is to be appreciated, that the memory 410 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 412 can be any of a variety of devices to receive input and/or provide output. For example, I/O devices 412 can include, a keyboard, a mouse, a joystick, a foot pedal, a display, a touch enabled display, a haptic feedback device, an LED, or the like.

Network interface 414 can include logic and/or features to support a communication interface. For example, network interface 414 may include one or more interfaces that operate according to various communication protocols or standards to communicate over direct or network communication links. Direct communications may occur via use of communication protocols or standards described in one or more industry standards (including progenies and variants). For example, network interface 414 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 414 can include logic and/or features to enable communication over a variety of wired or wireless network standards (e.g., 502.11 communication standards). For example, network interface 414 may be arranged to support wired communication protocols or standards, such as, Ethernet, or the like. As another example, network interface 414 may be arranged to support wireless communication protocols or standards, such as, for example, Wi-Fi, Bluetooth, ZigBee, LTE, 5G, or the like.

The intravascular imaging system acquisition circuitry 416 may include circuity including custom manufactured or specially programmed circuitry configured to receive or receive and send signals between intravascular imaging system 402 including indications of an IVUS run, a series of IVUS images, or a frame or frames of IVUS images.

Memory 410 can include instructions 418. During operation processor 408 can execute instructions 418 to cause computing device 402 to receive (e.g., from an IVUS imaging system of intravascular imaging systems 404, or the like) a series of IVUS images from a “run” through a vessel and store the recording as IVUS images 420 in memory 410. For example, processor 408 can execute instructions 418 to receive information elements from intravascular imaging systems 404 comprising indications of IVUS images captured by catheter 102 while being pulled back from distal end 204 to proximal end 206, which images comprise indications of the anatomy and/or structure of vessel 202 including vessel walls and plaque. It is to be appreciated that IVUS images 420 can be stored in a variety of image formats or even non-image formats or data structures that comprise indications of vessel 202. Further, IVUS images 420 will include several “frames” or individual images that, when represented co-linearly can be used to form an image or representation of the vessel 202, such as, for example, as represented by IVUS images 300a.

The present disclosure provides to generate and extract non-IVUS image modality features from IVUS images 420. For example, processor 408 can execute instructions 418 to generate non-IVUS image features 422 from IVUS images 420. Further, the present disclosure provides that non-IVUS image features 422 can be adapted the IVUS imaging modality and integrated into IVUS images 420 to form IVUS images adapted with non-IVUS image features 424. Processor 408 can execute instructions 418 to adapt the non-IVUS image features 422 to the IVUS imaging modality and generate IVUS images adapted with non-IVUS image features 424. In general, a machine learning (ML) model 426 can be used to infer IVUS images adapted with non-IVUS image features 424 from IVUS images 420. It is noted that although non-IVUS image features 422 are depicted and described as being generated and stored in memory 410, with some embodiments, ML model 426 can be trained and configured to generate IVUS images adapted with non-IVUS image features 424 from IVUS images 420 and non-IVUS image features 422 may be inferred during the process but not generated independently or stored in memory 410.

Further, processor 408 can execute instructions 418 to generate GUI 428 comprising indications of IVUS images adapted with features from non-IVUS images 424 and cause the GUI 428 to be displayed on display 406.

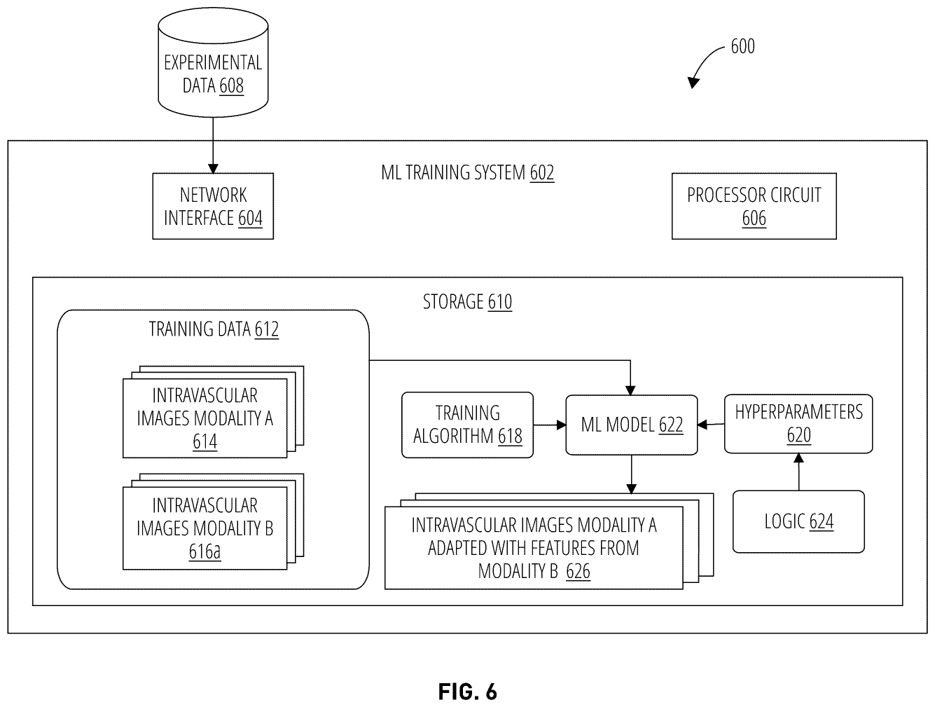

It is noted that although a single ML model 426 is depicted, some embodiments may provide ML model 426 having a model architecture and structure encompassing multiple discrete ML models. This will be described in greater detail below (e.g., FIG. 6 and beyond) where the balance of the disclosure turns to various systems and methodologies to train ML model 426, which can include deep generative models training methodologies.

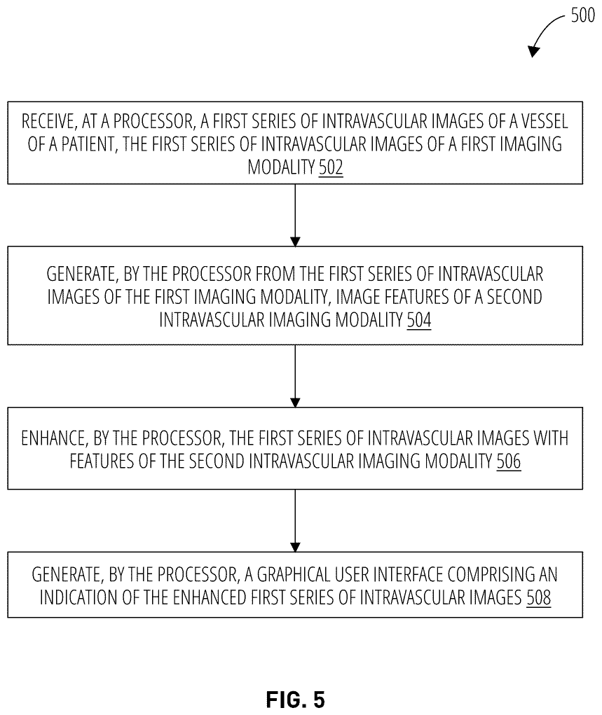

FIG. 5 illustrates a logic flow 500 to generate and extract from images of one imaging modality, features from another imaging modality and adapt them to the imaging modality and form a composite image from images and the adapted features, according to some embodiments of the present disclosure. The logic flow 500 can be implemented by multi-image modality adaptation system 400 and will be described with reference to multi-image modality adaptation system 400 for clarity of presentation. However, it is noted that logic flow 500 could also be implemented by an intravascular imaging system different than multi-image modality adaptation system 400. Further, as noted above, the examples provide to generate, extract, and adapt non-IVUS images features from IVUS images and generate a composite IVUS images comprising the original IVUS images and the adapted non-IVUS image features. However, this is not to be limiting, for example, the disclosure could be implemented to generate, extract, and adapt IVUS image features from non-IVUS images and generate a composite image comprising the adapted IVUS image features and the non-IVUS images.

Logic flow 500 can begin at block 502. At block 502 “receive, at a processor, a first series of intravascular images of a vessel of a patient, the first series of intravascular images of a first imaging modality” a first series of intravascular images of a vessel of a patient can be received. For example, logic flow 500 at block 502 can receive, at a processor, images captured via an IVUS catheter percutaneously inserted in a vessel of a patent. Processor 408 can execute instructions 418 to receive information elements comprising indications of IVUS images 420 from intravascular imaging systems 404, which can comprise an IVUS catheter (e.g., catheter 102, or the like) as the IVUS catheter is (or was) percutaneously inserted into vessel 202. It is to be appreciated that IVUS images 420 can comprise frames of images representative of images captured while the catheter 102 is pulled back from distal end 204 to proximal end 206. Processor 408 can execute instructions 418 to receive information elements comprising indications of IVUS images 420 from intravascular imaging systems 404, or directly from catheter 102 as may be the case.

Continuing to block 504 “generate, by the processor from the first series of intravascular images of the first imaging modality, image features of a second intravascular imaging modality” image features of a second intravascular imaging modality can be generated from a series of intravascular images of a first imaging modality. For example, logic flow 500 at block 504 can generate, by a processor, non-IVUS image features (e.g., OCT imaging modality features, or the like) from the series of IVUS images received at block 502. Processor 408 can execute instructions 418 to generate non-IVUS image features 422 from IVUS images 420.

Continuing to block 506 “enhance, by the processor, the first series of intravascular images with features of the second intravascular imaging modality” the first series of intravascular images can be enhanced with features from the second intravascular imaging modality. For example, processor 408 can execute instructions 418 to generate IVUS images adapted with non-IVUS image features 424 based on IVUS images 420 and non-IVUS image features 422. With some embodiments, processor 408 can execute instructions 418 to cause ML model 426 to infer IVUS images adapted with non-IVUS image features 424 from IVUS images 420 and/or non-IVUS image features 422. It is noted, in some embodiments, blocks 504 and 506 can be combined or rather, ML model 426 can be used to infer IVUS images adapted with non-IVUS image features 424 directly from IVUS images 420 and generation of non-IVUS image features 422 may be invisible or transparent to the user or computing system executing the ML model 426. For example, in some embodiments, ML model 426 can be configured to generate (e.g., infer, or the like) non-IVUS images from IVUS images and the translate features from the generated non-IVUS images to the IVUS image domain. Further, ML model 426 can be configured to integrate the translated features into the IVUS images.

Continuing to block 508 “generate a graphical user interface comprising an indication of the enhanced first series of intravascular images” a GUI can be generated where the GUI comprises graphical indications of the enhanced first series of intravascular images. For example, processor 408 can execute instructions 418 to generate GUI 428 as discussed above. As a specific example, processor 408 can execute instructions 418 to generate GUI 428, which includes graphical indications of IVUS images adapted with features from non-IVUS images 424.

As noted, with some embodiments, processor 408 of computing device 402 can execute instructions 418 to generate IVUS images adapted with non-IVUS image features 424 using ML model 426. In general, the present disclosure provides to use deep generative models to generate and extract non-IVUS imaging modality features from IVUS images 420 and adapt and integrate the extracted features into the IVUS images 420. The disclosure provides several embodiments of training a model (e.g., ML model 426) using deep generative models (e.g., training with paired and/or co-registered data, training with unpaired data, or the like). These different approaches are covered independently. However, it is to be appreciated that concepts from one training approach could be combined with concepts from another training approach. Further, although several training approaches are described in detail below, it will be appreciated that ML model 426 could be developed and trained using an approach not detailed herein.

To that end, FIG. 6 illustrates an ML training environment 600, according to various embodiments of the present disclosure. ML training environment 600 can be implemented to train an ML model to translate intravascular images from one modality to another (e.g., IVUS to OCT, OCT to IVUS, or the like). The ML training environment 600 may include an ML training system 602, such as a computing device that applies an ML algorithm to learn relationships between an input and an inferred output. The ML training system 602 may make use of experimental data 608 gathered during several prior procedures. Experimental data 608 can include intravascular images of a first (e.g., IVUS) and a second (e.g., OCT) modality from several patients. The experimental data 608 may be collocated with the ML training system 602 (e.g., stored in a storage 610 of the ML training system 602), may be remote from the ML training system 602 and accessed via a network interface 604, or may be a combination of local and remote data.

Experimental data 608 can be used to form training data 612. As noted above, the ML training system 602 may include a storage 610, which may include a hard drive, solid state storage, and/or random access memory. The storage 610 may hold training data 612. In general, training data 612 can include information elements or data structures comprising indications of intravascular images modality A 614 and intravascular images modality B 616a. In some embodiments, intravascular images modality A 614 can be paired with intravascular images modality B 616a. In other embodiments, intravascular images modality A 614 and intravascular images modality B 616a can be un-paired. As used herein, the term “paired” it intended to mean that the images intravascular images modality A 614 are co-registered with intravascular images modality B 616a. That is, intravascular images modality A 614 and intravascular images modality B 616a can include a series of images (e.g., set of frames) for multiple patients where each series of images includes multiple frames. The frames in a series of images in intravascular images modality A 614 for each patient can be co-registered (e.g., mapped or paired) with the frames in the series of images in intravascular images modality B 616a for each respective patient.

As noted, the present disclosure contemplates deep generative models such as GANs or deep diffusion models to train ML models. In general, the GAN framework consists of 2 networks, a generative network that transforms a noise vector into realistic samples and a discriminator network that classifies samples as real or fake. The training process of these 2 networks is a minimax game as the objective of the generator is to “fool” the discriminator. GANs can be applied to translate an image from one domain to another. As such, in general, ML model 622 will comprise multiple networks. Depending on the application, different types of models may be used to form the basis of the networks in ML model 622. For instance, in the present example, an artificial neural network (ANN) or a convolutional neural network (CNN) may be particularly well-suited to learning associations between images of different domains as detailed herein.

Any suitable training algorithm 618 may be used to train the models within ML model 622. Nonetheless, the example depicted in FIG. 6 may be particularly well-suited to a supervised training algorithm or unsupervised training algorithm. For a supervised training algorithm, the ML training system 602 may apply the training data 612 as inputs with their annotations, to which an expected output (e.g., intravascular images modality A adapted with features from modality B 626) can be generated by ML model 622 With some embodiments, training data 612 can be split into “training” and “testing” data wherein some subset of the training data 612 can be used to adjust the networks in ML model 622 (e.g., internal weights of the model, or the like) while another, non-overlapping subset of the training data 612 can be used to measure an accuracy of the ML model 622 to infer (or generalize) an output from “unseen” inputs.

The networks in ML model 622 may be applied using a processor circuit 606, which may include suitable hardware processing resources that operate on the logic and structures in the storage 610. The training algorithm 618 and/or the development of the trained ML model 622 may be at least partially dependent on hyperparameters 620. In exemplary embodiments, the model hyperparameters 620 may be automatically selected based on logic 624, which may include any known hyperparameter optimization techniques as appropriate to the ML model 622 selected and the training algorithm 618 to be used. In optional, embodiments, the ML model 622 may be re-trained over time, to accommodate new knowledge and/or updated experimental data 608.

Further, as noted, ML model 622 can be a GAN. As such, network architectures and training algorithms specific to GANs can be employed. The details of such network architectures and training algorithms are discussed below. However, other details can be provided by those skill in the art. Once the ML model 622 is trained, it may be applied (e.g., by the processor 408, or the like) to new input data (e.g., intravascular images of different modalities). This input to the ML model 622 may be formatted mirroring the way that the training data 612 was provided to the ML model 622. The ML model 622 may generate an output (e.g., intravascular images of one modality augmented with features from another modality as discussed herein.

Further, as noted, ML model 622 can be a generative diffusion model. As such, network architectures and training algorithms specific to generative diffusion models can be employed. Diffusion models are part of deep generative models that involves a forward diffusion stage and a reverse denoising stage. Forward diffusion stage gradually alters the input image by adding Gaussian noise iteratively. In the reverse denoising stage, a model learns to recover the input image by iteratively reversing the diffusion process. A recent published survey on generative diffusion models on images describes the details of applications: Croitoru F A, Hondru V, Ionescu R T, Shah M. Diffusion models in vision: A survey. IEEE Transactions on Pattern Analysis and Machine Intelligence. March 2023, which article is incorporated herein by reference in its entirety.

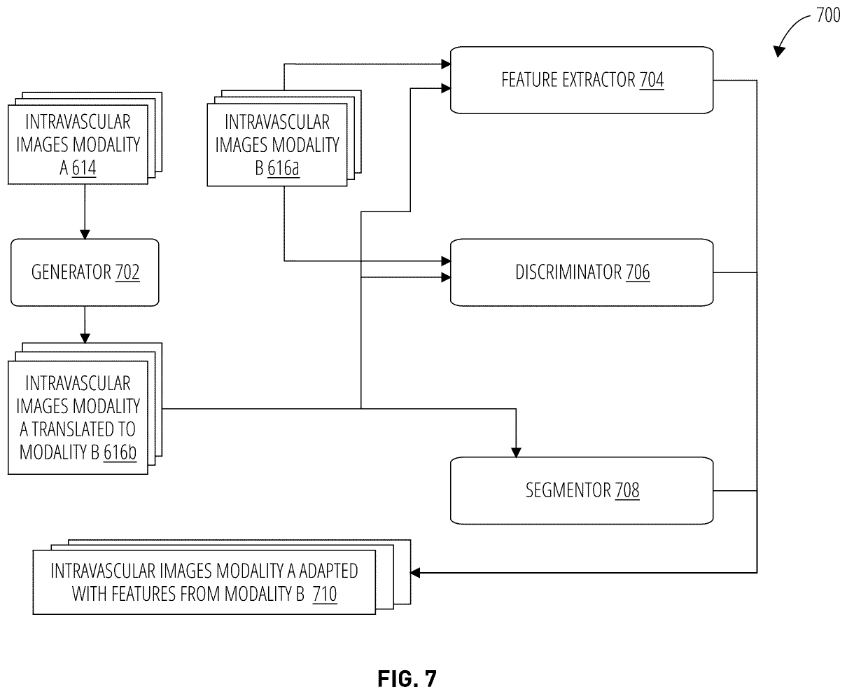

FIG. 7 illustrates an ML model architecture 700, according to some embodiments of the present disclosure. With some embodiments, ML model architecture 700 can be provided as ML model 622 or ML model 426. In general, ML model architecture 700 utilizes a MedGAN architecture for image to image translation in addition with hybrid ground truth mask segmentation networks and trains these networks as an auxiliary task to preserve the morphologically important structures in the translated images, thus generating enhanced image features from translated images. An example MedGAN network is described in MedGAN: Medical Image Translation using GANs, published in the Journal of Latex Class Files, Vol. 14, No. 8, August 2015, which article is incorporated herein by reference in its entirety.

ML model architecture 700 can include a generator network 702 trained to receive as input intravascular images of first modality (e.g., intravascular images modality A 614, IVUS images, or the like) and output will be images of a second, different modality, that look like the images of the first modality (e.g., intravascular images modality A translated to modality B 616b, OCT looking IVUS images, or the like). Accordingly, given a source domain D_{S}=(X^{S}, Y) and a target domain D^{T}=(X^{T}), the input to generator network 702 can be defined as y and the output as \hat{x}. In some examples, generator network 702 has a generator architecture arranged to generate as \hat{x} (e.g., intravascular images modality A translated to modality B 616b) from y (e.g., intravascular images modality A 614) through progressive refinement via encoder decoder blocks.

ML model architecture 700 can further include a feature extractor network 704, a discriminator network 706, and a segmentor network 708 arranged to calculate style and content loss, perceptual and adversarial loss, and segmentation loss respectively. Those five losses form the loss function to update model weights through back-propagation. Discriminator network 706 is trained to discriminate between intravascular images modality B 616a and intravascular images modality A translated to modality B 616b and generate adversarial loss as well as serve as a trainable feature extractor whose hidden layers are used to calculate the modified perceptual loss.

Feature extractor network 704 is trained to extract and transfer style and content from the intravascular image modality B 616a and intravascular images modality A translated to modality B 616b into intravascular images modality A adapted with features from modality B 710. Feature extractor network 704 is used to extract deep rich features Vᵢ(\hat{x}) to calculate style transfer losses in order for the output to match the target's style, textures, and content.

Segmentor network 708 is trained to segment features from the images translated from a source modality (e.g., IVUS) and combine them with the images of the target modality (e.g., OCT). As such, ML model architecture 700 provides a ML architecture where features from multiple imaging modalities can be combined to preserve modality specific features (e.g., IVUS-based lumen/vessel borders and OCT-based calcium segmentation masks, or the like). Segmentor network 708 provides for preserving geometry of the features as they are translated, extracted, and combined into intravascular images modality A adapted with features from modality B 710. In particular, the input to segmentor network 708 can be defined as (\hat{x}, x) and the output as (ŷ^{s2}, ŷ^{t2}).

It is contemplated that ML model architecture 700 will utilize non-adversarial loss from auxiliary tasks (e.g., segmentor network 708) during training of the generator network 702. Accordingly, a loss function for ML model architecture 700 can be formulated as:

$${700}_{} = {λ}_{1} {706 \_ Adversarial}_{} + {λ}_{1} {706 \_ Precept}_{} + {λ}_{2} {704 \_ Style}_{} + {λ}_{3} {704 \_ Content}_{} + ( {{708}_{}}^{( {\hat{Y}}^{S 3} , {Y}^{S + T} )} + {{708}_{}}^{( {\hat{Y}}^{T 2} , {Y}^{S + T} )} )$$

where λ₁, λ₂, and λ₃ are hyperparamters.

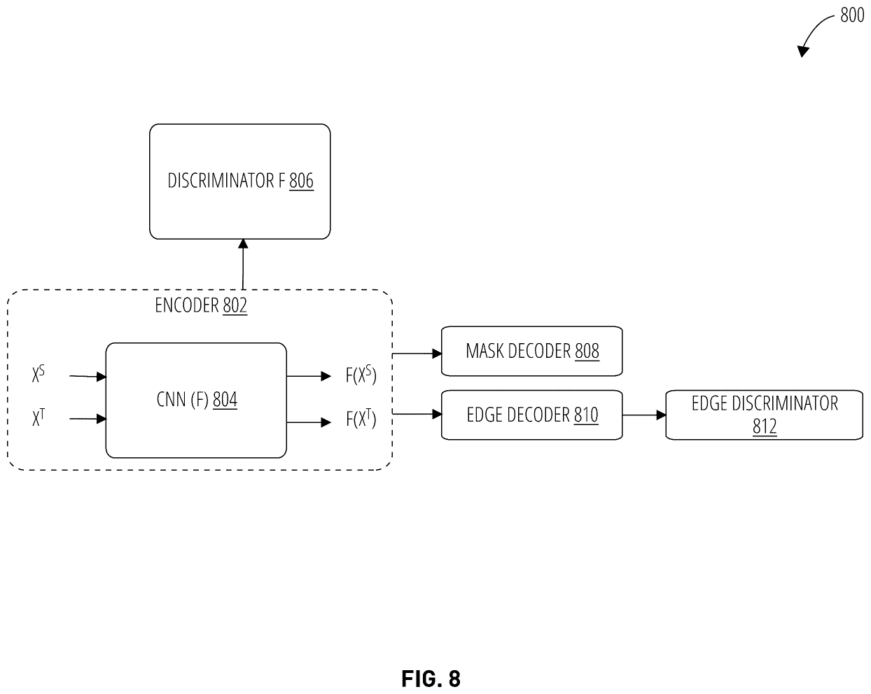

FIG. 8 illustrates an ML model architecture 800, according to some embodiments of the present disclosure. With some embodiments, ML model architecture 800 can be provided as ML model 622 or ML model 426. In general, ML model architecture 800 utilizes a convolutional neural network (CNN) encoder trained, with adversarial loss, to build a domain invariable embedding space, which can be used to translate information from a source image modality to a target image modality. In general, the input to ML model architecture 800 will be images from a first image modality and the output will be an enhanced segmentation mask with features adapted from the first image modality and a second image modality.

ML model architecture 800 can include an encoder 802 which itself is a CNN 804 arranged to take as first and second source data (X^{s}, Y^{s}) and (X^{t}, Y^{t}), where X^{s} is images of a first image modality (e.g., OCT) and Y^{s} is a segmentation mask for the image of the first image modality (e.g., calcium mask, or the like) and where X^{t} is images of a second image modality (e.g., IVUS) and Y^{t} is a segmentation mask for the image of the second image modality (e.g., lumen/vessel borders, or the like). Accordingly, encoder 802 is configured to learn the domain invariant representations (F) of source and target images. ML model architecture 800 further includes discriminator F 806 which can be a classifier network configured to classify the domain of the encoded input F(x).

Additionally, ML model architecture 800 provides a segmentation network and an auxiliary edge detection network along with a discriminator to train the network based on adversarial and non-adversarial losses. ML model architecture 800 includes mask decoder 808, edge decoder 810, and edge discriminator 812. Mask decoder 808 is configured to generate hybrid segmentation masks (ŷ^{s2}, ŷ^{t2}) while edge decoder 810 is configured to generate hybrid edge masks (ê^{s2}, ê^{t2}). Further, edge discriminator 812 is configured to classify the domain of edge map output by edge decoder 810 (e.g., D_{E}(F(X))).

With some embodiments, ML model architecture 800 can be trained with an objective function L_{H}=L_{CE}^{y}+λ₁L_{F}+L_{CE}^{e}−λ₂L_{E}, where L_{F} is L_{F}(F(X^{s}),F(X^{t})) and is associated with discriminator F 806, L_{CE}^{y} is L_{CE}^{(ŷ}^{^{s2}}^{,y}^{^{s+t}}⁾+L_{CE}^{(ŷ}^{^{t2}}^{,y}^{^{s+t}}⁾ and L_{CE}^{e} is L_{CE}^{(ê}^{^{s2}}^{,e}^{^{s+t}}⁾+L_{CE}^{(ê}^{^{t2}}^{,e}^{^{s+t}}⁾, and L_{CE}^{e} , and both of which are associated with mask decoder 808; and L_{E} is L_{E}^{(ê}^{^{s2}}^{,ê}^{^{t2}}⁾ and is associated with edge discriminator 812; and where λ₁ and λ₂ are hyperparamters. It will be appreciated that ML model architecture 800 is arranged to be trained with paired images of different modalities with ground truth masks y^{s+t} and e^{s+t}.

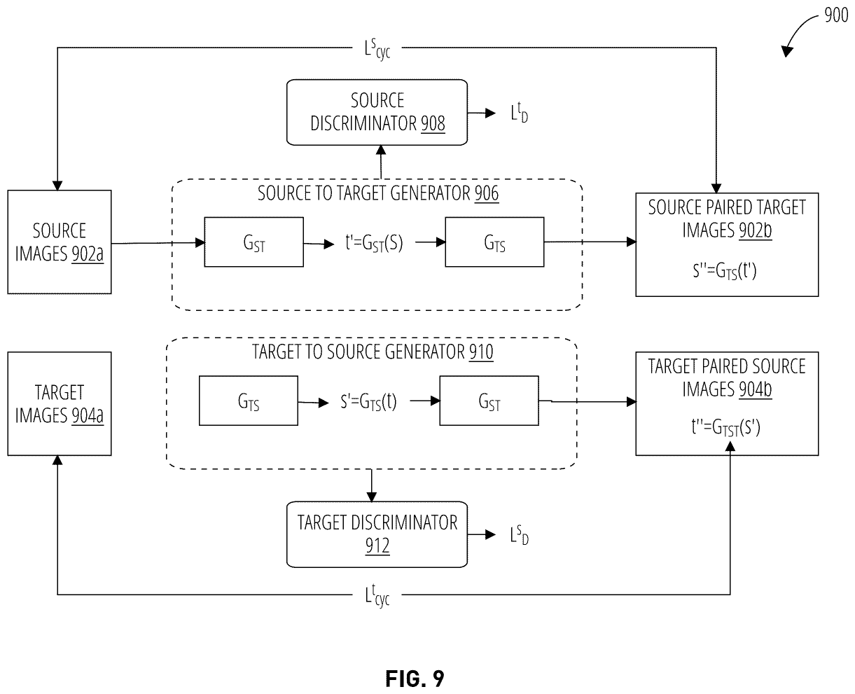

FIG. 9 illustrates an ML model architecture 900, according to some embodiments of the present disclosure. With some embodiments, ML model architecture 900 can be provided as ML model 622 or ML model 426. In general, ML model architecture 900 utilizes a cycle generative adversarial network (cycle-GAN). In general, a cycle-GAN network consists of two generators and two discriminators, which perform image translation between two image modalities without using paired source and target images. This provides bi-directional image translation between source and target domains. One of the generators translates source images into target images and a discriminator distinguishes generated target image and real target image. The second generator will translate target domain images to source domain images and a second discriminator which will classify the generated source images as either real or fake.

In this manner, the generators will try to generate more realistic images against to discriminator. Cycle-consistency loss, adversarial losses and non-adversarial losses can be used to train a cycle-GAN network. It is to be appreciated that ML model architecture 900 can be implemented to train a network for generating hybrid images comprising indications from multiple image modalities where paired images are not available for training the network.

ML model architecture 900 can operate on unpaired source images 902a (e.g., IVUS images, OCT images, or the like) and target images 904a (e.g., OCT images, IVUS images, or the like) where source images 902a and target images 904a are images of different domains or modalities. ML model architecture 900 can comprise source to target generator 906 and source discriminator 908 as well as target to source generator 910 and target discriminator 912.

Source to target generator 906 can be configured to generate source paired target images 902b from source images 902a while target to source generator 910 can be configured to generate target paired source images 904b from a target images 904a. Source discriminator 908 and target to source generator 910 can be configured to classify source paired target images 902b and target paired source images 904b, respectively, as real, or fake.

Hybrid label masks can be generated for both source images 902a and target images 904a to be used as a ground truth in training. Once ML model architecture 900 is trained, 900 can be utilized to generate images for training a network like ML model architecture 800. As such, the objective function for ML model architecture 900 can be defined as:

$${L}_{Total} = {L}_{Network \_ 800} + {L}_{cyc} + {L}_{D}^{t} + {L}_{D}^{s} ,$$

$$where {L}_{D}^{t} = {E}_{t} [ log {D}_{t} ( t ) ] + {E}_{s} [ log ( 1 - {D}_{t} ( {G}_{ST} ( S ) ) ) ] ,$$

$${L}_{D}^{s} = {E}_{s} [ log {D}_{s} ( s ) ] + {E}_{t} [ log ( 1 - {D}_{t} ( {G}_{TS} ( t ) ) ) ] , {L}_{cyc}^{t} = {E}_{t} [ {‖ t - t ″ ‖}_{1} ] ,$$

$${L}_{cyc}^{s} = {E}_{s} [ {‖ s - s ″ ‖}_{1} ] , {L}_{cyc} = {L}_{cyc}^{s} + {L}_{cyc}^{t} , and$$

$${L}_{Network \_ 800} = {L}_{CE}^{y} - {λ}_{1} {L}_{F} + {L}_{CE}^{e} - {λ}_{2} {L}_{E}$$

as defined above with respect to ML model architecture 800.

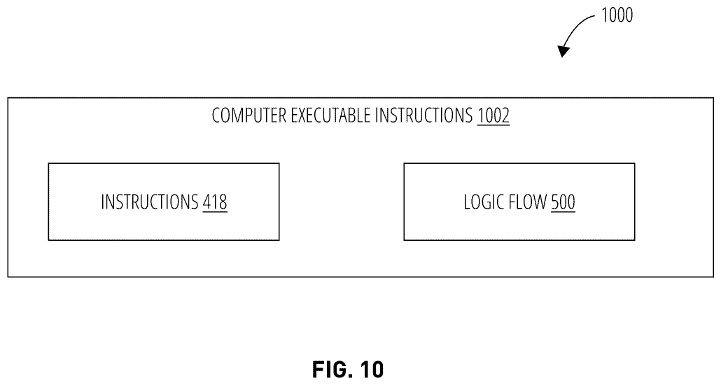

FIG. 10 illustrates computer-readable storage medium 1000. Computer-readable storage medium 1000 may comprise any non-transitory computer-readable storage medium or machine-readable storage medium, such as an optical, magnetic or semiconductor storage medium. In various embodiments, computer-readable storage medium 1000 may comprise an article of manufacture. In some embodiments, computer-readable storage medium 1000 may store computer executable instructions 1002 with which circuitry (e.g., processor 106, processor 408, and the like) can execute. For example, computer executable instructions 1002 can include instructions to implement operations described with respect to instructions 418 and/or logic flow 500. Examples of computer-readable storage medium 1000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1002 may include any suitable type of code, such as source code, compiled code, interpreted code, executable code, static code, dynamic code, object-oriented code, visual code, and the like.

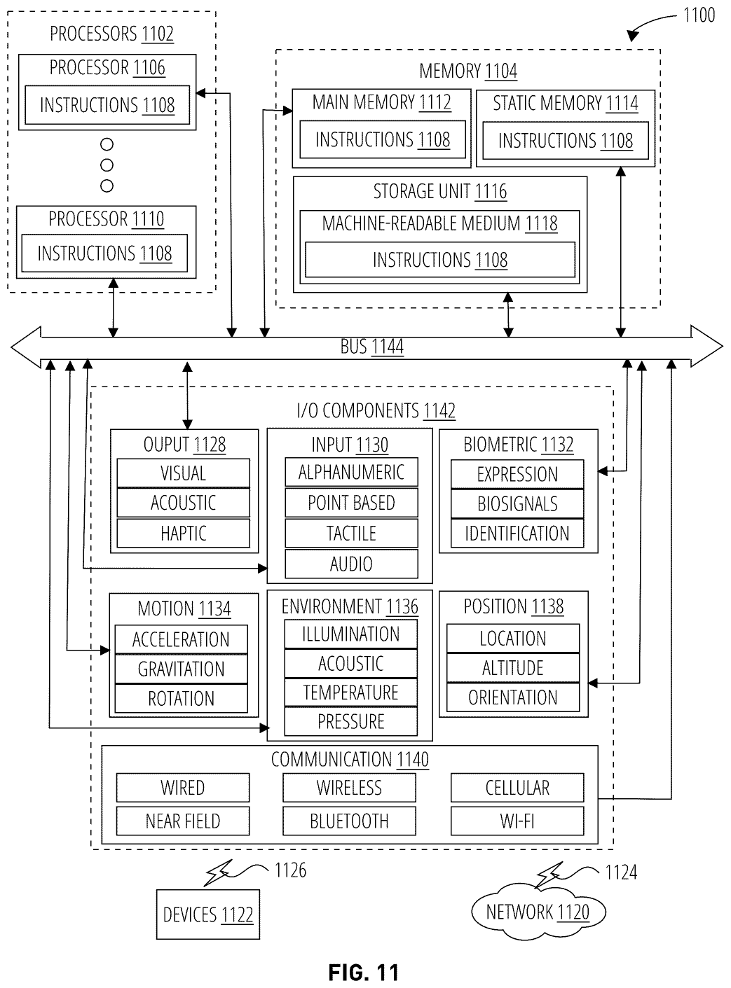

FIG. 11 illustrates a diagrammatic representation of a machine 1100 in the form of a computer system within which a set of instructions may be executed for causing the machine to perform any one or more of the methodologies discussed herein. More specifically, FIG. 11 shows a diagrammatic representation of the machine 1100 in the example form of a computer system, within which instructions 1108 (e.g., software, a program, an application, an applet, an app, or other executable code) for causing the machine 1100 to perform any one or more of the methodologies discussed herein may be executed. For example, the instructions 1108 may cause the machine 1100 to execute logic flow 500 of FIG. 5, or the like. More generally, the instructions 1108 may cause the machine 1100 to extract features from images of a first modality and integrate them into images of a second modality.

The instructions 1108 transform the general, non-programmed machine 1100 into a particular machine 1100 programmed to carry out the described and illustrated functions in a specific manner. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1108, sequentially or otherwise, that specify actions to be taken by the machine 1100. Further, while only a single machine 1100 is illustrated, the term “machine” shall also be taken to include a collection of machines 1100 that individually or jointly execute the instructions 1108 to perform any one or more of the methodologies discussed herein.

The machine 1100 may include processors 1102, memory 1104, and I/O components 1142, which may be configured to communicate with each other such as via a bus 1144. In an example embodiment, the processors 1102 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1106 and a processor 1110 that may execute the instructions 1108. The term “processor” is intended to include multi-core processors that may comprise two or more independent processors (sometimes referred to as “cores”) that may execute instructions contemporaneously. Although FIG. 11 shows multiple processors 1102, the machine 1100 may include a single processor with a single core, a single processor with multiple cores (e.g., a multi-core processor), multiple processors with a single core, multiple processors with multiples cores, or any combination thereof.

The memory 1104 may include a main memory 1112, a static memory 1114, and a storage unit 1116, both accessible to the processors 1102 such as via the bus 1144. The main memory 1104, the static memory 1114, and storage unit 1116 store the instructions 1108 embodying any one or more of the methodologies or functions described herein. The instructions 1108 may also reside, completely or partially, within the main memory 1112, within the static memory 1114, within machine-readable medium 1118 within the storage unit 1116, within at least one of the processors 1102 (e.g., within the processor's cache memory), or any suitable combination thereof, during execution thereof by the machine 1100.

The I/O components 1142 may include a wide variety of components to receive input, provide output, produce output, transmit information, exchange information, capture measurements, and so on. The specific I/O components 1142 that are included in a particular machine will depend on the type of machine. For example, portable machines such as mobile phones will likely include a touch input device or other such input mechanisms, while a headless server machine will likely not include such a touch input device. It will be appreciated that the I/O components 1142 may include many other components that are not shown in FIG. 11. The I/O components 1142 are grouped according to functionality merely for simplifying the following discussion and the grouping is in no way limiting. In various example embodiments, the I/O components 1142 may include output components 1128 and input components 1130. The output components 1128 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1130 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1142 may include biometric components 1132, motion components 1134, environmental components 1136, or position components 1138, among a wide array of other components. For example, the biometric components 1132 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1134 may include acceleration sensor components (e.g., accelerometer), gravitation sensor components, rotation sensor components (e.g., gyroscope), and so forth. The environmental components 1136 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1138 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1142 may include communication components 1140 operable to couple the machine 1100 to a network 1120 or devices 1122 via a coupling 1124 and a coupling 1126, respectively. For example, the communication components 1140 may include a network interface component or another suitable device to interface with the network 1120. In further examples, the communication components 1140 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1122 may be another machine or any of a wide variety of peripheral devices (e.g., a peripheral device coupled via a USB).

Moreover, the communication components 1140 may detect identifiers or include components operable to detect identifiers. For example, the communication components 1140 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1140, such as location via Internet Protocol (IP) geolocation, location via Wi-Fi® signal triangulation, location via detecting an NFC beacon signal that may indicate a particular location, and so forth.

The various memories (i.e., memory 1104, main memory 1112, static memory 1114, and/or memory of the processors 1102) and/or storage unit 1116 may store one or more sets of instructions and data structures (e.g., software) embodying or utilized by any one or more of the methodologies or functions described herein. These instructions (e.g., the instructions 1108), when executed by processors 1102, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1120 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1120 or a portion of the network 1120 may include a wireless or cellular network, and the coupling 1124 may be a Code Division Multiple Access (CDMA) connection, a Global System for Mobile communications (GSM) connection, or another type of cellular or wireless coupling. In this example, the coupling 1124 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1108 may be transmitted or received over the network 1120 using a transmission medium via a network interface device (e.g., a network interface component included in the communication components 1140) and utilizing any one of several well-known transfer protocols (e.g., hypertext transfer protocol (HTTP)). Similarly, the instructions 1108 may be transmitted or received using a transmission medium via the coupling 1126 (e.g., a peer-to-peer coupling) to the devices 1122. The terms “transmission medium” and “signal medium” mean the same thing and may be used interchangeably in this disclosure. The terms “transmission medium” and “signal medium” shall be taken to include any intangible medium that can store, encoding, or carrying the instructions 1108 for execution by the machine 1100, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for an intravascular ultrasound (IVUS) imaging system, comprising a memory and a processor coupled to the memory and configured to couple to an IVUS probe, the memory comprising instructions executable by the processor, which instructions when executed by the processor cause the processor to:
receive a first series of intravascular images of a vessel of a patient, the first series of intravascular images of a first imaging modality;
generate, from the first series of intravascular images, image features of a second imaging modality;
enhance the first series of intravascular images with the image features of the second series imaging modality; and
generate a graphical user interface comprising an indication of the enhanced first series of intravascular images.

2. The apparatus of claim 1, the instructions when executed by the processor further cause the processor to cause the graphical user interface to be displayed on a display coupled to the computing device.

3. The apparatus of claim 1, the instructions when executed by the processor further cause the processor to:
generate, via a machine learning (ML) model, a second series of intravascular images of the vessel of the patient, the second series of intravascular images of the second imaging modality; and
generate, via the ML model, the image features of the second imaging modality from the first series of intravascular images.

4. The apparatus of claim 3, the instructions when executed by the processor further cause the processor to:
translate, via the ML model, the first series of intravascular images to the second imaging modality to form a series of translated intravascular images, wherein the series of translated intravascular image features look like image features of the second imaging modality; and
extract, via the ML model, features from the series of translated images.

5. The apparatus of claim 4, the instructions when executed by the processor further cause the processor to generate, via the ML model, a series of hybrid intravascular images comprising the first series of intravascular images and the image features of the second imaging modality.

6. The apparatus of claims 5, wherein the ML model comprises a medical image generative network and auxiliary task networks, wherein the auxiliary task networks are arranged to preserve the geometry of extracted features.

7. The apparatus of claim 6, wherein the ML model is trained using a plurality of series of intravascular images of the first modality paired or unpaired with a respective series of a plurality of series of intravascular images of the second modality.

8. The apparatus of claim 7, wherein the medical image generative model is trained with non-adversarial loss from the auxiliary task network.

9. The apparatus of claim 3, wherein the ML model comprises a convolutional neural network (CNN) based encoder network and a first decoder network and a second decoder network.

10. The apparatus of claim 9, wherein the CNN based encoder network is arranged to translate a series of intravascular images of the first imaging modality into a series of intravascular images of the second imaging modality and translate a series of intravascular images of the second imaging modality into a series of intravascular images of the first imaging modality.

11. The apparatus of claim 10, wherein the first decoder network is arranged to extract features from the series of intravascular images translated from the first imaging modality.

12. The apparatus of claim 11, wherein the second decoder network is arranged to extract features from the series of intravascular images translated from the second imaging modality.

13. The apparatus of claim 9, wherein the ML model is trained with a plurality of series of intravascular images of the first modality paired or unpaired with a respective series of a plurality of series of intravascular images of the second modality annotated with ground truth masks.

14. At least one machine readable storage device, comprising a plurality of instructions that in response to being executed by a processor of an intravascular ultrasound (IVUS) imaging system cause the processor to:
receive, at the processor, a first series of intravascular images of a vessel of a patient, the first series of intravascular images of a first imaging modality;
generate, by the processor from the first series of intravascular images, image features of a second imaging modality;
enhance, by the processor, the first series of intravascular images with the image features of the second series imaging modality; and
generate, by the processor, a graphical user interface comprising an indication of the enhanced first series of intravascular images.

15. The at least one machine readable storage device of claim 14, the instructions when executed by the processor further cause the processor to cause the graphical user interface to be displayed on a display coupled to the computing device.

16. The at least one machine readable storage device of claim 14, the instructions when executed by the processor further cause the processor to:
generate, via a machine learning (ML) model, a second series of intravascular images of the vessel of the patient, the second series of intravascular images of the second imaging modality; and
generate, via the ML model, the image features of the second imaging modality from the first series of intravascular images.

17. The at least one machine readable storage device of claim 3, the instructions when executed by the processor further cause the processor to:
translate, via the ML model, the first series of intravascular images to the second imaging modality to form a series of translated intravascular images, wherein the series of translated intravascular image features look like image features of the second imaging modality; and
extract, via the ML model, features from the series of translated images.

18. A method for a computing device, comprising:
receiving, at a processor, a first series of intravascular images of a vessel of a patient, the first series of intravascular images of a first imaging modality;
generating, by the processor from the first series of intravascular images, image features of a second imaging modality;
enhancing, by the processor, the first series of intravascular images with the image features of the second series imaging modality; and
generating, by the processor, a graphical user interface comprising an indication of the enhanced first series of intravascular images.

19. The method of claim 18, comprising causing the graphical user interface to be displayed on a display coupled to the computing device.

20. The method of claim 18, generating the image features of a second imaging modality comprising:
generating, via a machine learning (ML) model, a second series of intravascular images of the vessel of the patient, the second series of intravascular images of the second imaging modality;
generating, via the ML model, the image features of the second imaging modality from the first series of intravascular images;
translating, via the ML model, the first series of intravascular images to the second imaging modality to form a series of translated intravascular images, wherein the series of translated intravascular image features look like image features of the second imaging modality; and
extracting, via the ML model, features from the series of translated images.
